(19) United States
(12) Patent Application Publication
(10) Pub. No.: US 2019/0117057 A1
(43) Pub. Date: Apr. 25, 2019

(54) INTEGRATED PROBE AND ANOSCOPE FOR ELECTRICAL CURRENT THERAPY OF TISSUE

(71) Applicant: ULTROID, LLC, Tampa, FL (US)

(72) Inventor: Michael Knox, Tampa, FL (US)

(21) Appl. No.: 16/090,474

(22) PCT Filed: Mar. 28, 2017
(86) PCT No.: PCT/US2017/024627

§ 371 Date: Oct. 1, 2018

Related U.S. Application Data
(60) Provisional application No. 62/314,769, filed on Mar. 29, 2016.

(51) Int. Cl. A61B 1/31; A61B 1/32; A61B 18/14

(52) U.S. Cl. CPC A61B 1/31; A61B 2018/005; A61B 18/14; A61B 1/32

(57) ABSTRACT

Disclosed herein is a surgical tool (10′, 10″) for use in surgical removal of hemorrhoidal tissue (50). The surgical tool includes an anoscope (51′, 51″) with a proximal end (55′, 55″) and a distal end (57). The anoscope (51′ 51″) has a slot (53) near the distal end (57) to receive the hemorrhoidal tissue (50). The surgical tool also includes a probe (16) for delivering electrical current to the hemorrhoidal tissue (50). The probe (16) includes a base (48) with a distal end and a proximal end and elongated probe electrodes (36, 38) extending from the distal end of the base. The probe (16) is removably attached to the anoscope (51′, 51″) such that distal ends (37, 39) of the elongated probe electrodes (36, 38) are positioned within the slot (53) for electricity-conducting engagement with said hemorrhoidal tissue (50).

### BACKGROUND

The invention relates to a novel surgical tool including an integrated probe and anoscope designed for electrical current therapy to treat hemorrhoids.

Hemorrhoidal disease is one of the most frequent, disabling, and painful conditions of mankind. The consensus of the Advisory Panel of the U.S. Food and Drug Administration defines hemorrhoids as “abnormally large or symptomatic conglomerates of blood vessels, supporting tissues, and overlying mucous membrane or skin of the anorectal area.”

It is estimated that one-third of the U.S. population has symptomatic internal hemorrhoids, with an incidence of 50% at age 50 years. Patients frequently postpone examination because of concern of pain associated with a particular treatment modality, hospitalization, cost, and time of disability. Such a delay in evaluation may lead to progression of the hemorrhoidal disease, or late diagnosis of more serious colorectal problems.

FIGS. 1A-1B depict a conventional surgical tool 10 including a separate probe 16 and anoscope 51 for electrical current therapy to treat hemorrhoids 50. The probe 16 is mounted to a distal end 44 of a handpiece 14, by inserting an end of the probe 16 in an aperture 40 in the distal end 44 of the handpiece 14. The handpiece 14 consists of a handle portion 18 from which a connector cord 20 extends to supply electrical current to the probe 16 from a base unit (not shown). The handpiece 14 also includes a planar face surface 23 that contains display elements of, e.g., elapsed time of treatment (LED numeric display 28), level of treatment current (LED bar graph 26), and circuit status indicator (on/off display 30). The planar face surface 23 further contains switches 32, 34 for respectively incrementally increasing and decreasing the level of D.C. current through the probe 16 and, when activated by the surgeon 52 simultaneously, e.g., by pressing with his thumb, ceases flow of electricity. The handpiece face surface 23 is sealed against entry of fluid into the handpiece 14 during cleaning between procedures.

The probe 16 consists of a pair of elongated, electrically-conductive electrodes 36, 38 extending to sharpened distal ends. The probe electrodes 36, 38 are clad in nonconductive sleeves over their distal portions to prevent inadvertent tissue contact, which minimizes obstruction to the surgeon's view of the treatment site 54. The proximal portions of the electrodes 36, 38 are encased in a base 48, e.g., of injection molded plastic. The probe base 48 is constructed to be received in the aperture 40 at four orientations (90° rotations), selected by the surgeon 52 depending upon the rectal quadrant to be treated. The distal end of one electrode 36 of the pair extends longitudinally beyond the distal end of the second electrode 38, whereby during treatment, the distal ends do not penetrate the tissue simultaneously, for reduced patient discomfort.

An axis, P, of the probe 16, in assembly with the handpiece 14, extends at an angle, A, preferably about 120°, relative to the plane of face surface 23 of the handpiece 14. During treatment of hemorrhoids 50, the distal ends of the electrodes 36, 38 are in contact with the hemorrhoidal tissue 50, and the important display elements on the planar face surface 23 are both immediately along a line of sight, L, of the surgeon 52, allowing him to constantly observe the treatment site 54 (i.e. hemorrhoidal tissue 50 in contact with electrode 36, 38 distal ends) and the treatment parameters on the planar face surface 23, without turning away from the patient 56.

During the electrical current therapy with the conventional surgical tool 10, the patient 56 is first positioned in a right lateral fetal position. The surgeon 52 then inserts and positions the anoscope 51 to expose the hemorrhoid 50 through the slot 53 to be treated. The surgeon 52 then activates the surgical tool 10 and the display 30 indicates that the circuit is not closed. As shown in FIGS. 1A-1B, the surgeon 52 positions one hand on the handle portion 18 of the handpiece 14 and the other hand on the anoscope 51. The surgeon 52 then adjusts the relative position between the anoscope 51 and the probe 16 (via. the handpiece 14), such that the distal ends of the electrodes 36, 38 engage the base of the hemorrhoidal lesion 50. The display 30 then indicates a closing of the circuit. The surgeon 52 then incrementally increases current through the probe 16 by depressing switch 32, with the level of current indicated by the bar graph 26, and the elapsed time of treatment indicated by display 28. As current is initiated, the surgeon 52 urges the tips of the electrodes 36, 38 into the base of the hemorrhoidal lesion 50.

It has been observed that the degree of treatment required for electrical current therapy of the hemorrhoid 50 is a function of time and current, i.e., the higher the level of current, the shorter the time required for each treatment procedure. A factor limiting current intensity is patient discomfort; the surgeon 52, therefore, by depressing switch 32, slowly increases the level of current as high as possible without patient discomfort (experienced as an aching feeling) in order to shorten the time of treatment. Should the patient 56 feel discomfort, or when the treatment is completed, the surgeon reduces the current incrementally by depressing switch 34. A factor limiting the time of treatment is the surgeons 52 ability to maintain contact between the distal ends of the electrodes 36, 38 and the base of the hemorrhoidal lesion 50. In order to maintain this contact, the surgeon 52 must maintain a fixed relative position between the handpiece 14 (held in one hand) and the anoscope 51 (held in the other hand).

### BRIEF DESCRIPTION OF THE DRAWINGS

FIG. 1A is a side view of a conventional surgical tool including a separate probe and anoscope for electrical current therapy to treat hemorrhoids;

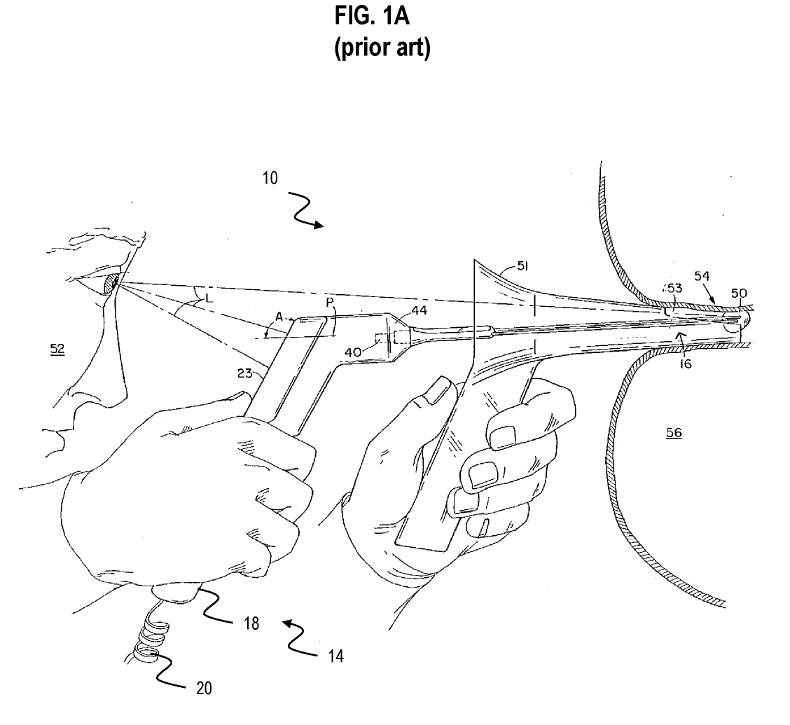

FIG. 1B is a rear perspective view of the conventional surgical tool of FIG. 1A;

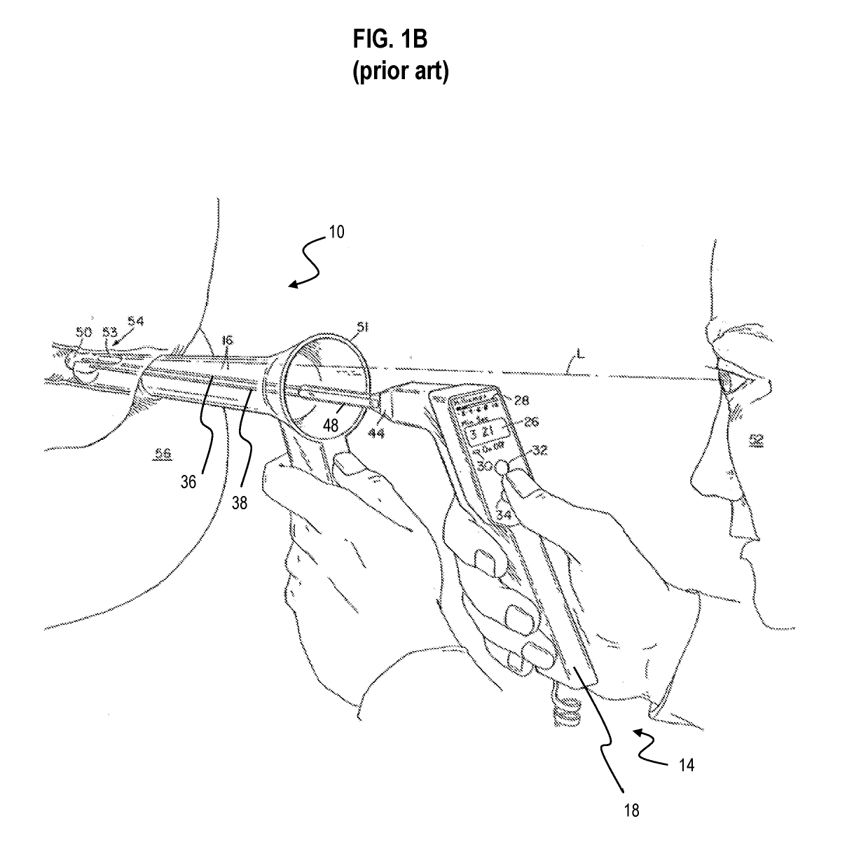

FIG. 2 is a side view of a surgical tool including an integrated probe and anoscope for electrical current therapy to treat hemorrhoids, according to one embodiment of the present invention;

FIG. 3 is a side view of the surgical tool of FIG. 2 during electrical current therapy by a medical professional;

FIG. 4 is a side view of a surgical tool including an integrated probe and anoscope for electrical current therapy to treat hemorrhoids, according to one embodiment of the present invention;

FIG. 5 is cross-sectional view of the surgical tool of FIG. 4;

FIG. 6 is a perspective view of the anoscope of the surgical tool of FIG. 4;

FIG. 7A is a front view of the handpiece of the surgical tool of FIG. 4; and

FIG. 7B is a side view of the handpiece of the surgical tool of FIG. 4.

### DETAILED DESCRIPTION

As previously discussed, a limiting factor of the conventional surgical tool 10 is that the surgeon 52 must maintain a fixed relative position between the handpiece 14 (held in one hand) and the anoscope 51 (held in the other hand), in order to maintain contact between the electrodes 36, 38 and the hemorrhoid 50. In the event that the surgeon 52 hands tire and he needs to rest his hands, the treatment time will increase, reducing the efficiency of the treatment. Additionally, another limiting factor of the conventional surgical tool 10 is the surgeons 52 ability to maintain the fixed relative position between the handpiece 14 and anoscope 51. In the event of undesired relative movement between the surgeons 52 hands, the electrodes 36, 38 could move from the hemorrhoid 50 and contact another part of the patient 56, resulting in unnecessary patient discomfort to that area. The inventor of the present invention realized that if the surgeon 52 is not required to maintain the fixed relative position between the handpiece 14 and anoscope 51, the treatment time would necessarily be reduced, resulting in more efficient treatment. The inventor of the present invention also realized that if the surgeon 52 is not required to maintain the fixed relative position between the handpiece 14 and anoscope 51, the risk of discomfort to others parts of the patient 56 would be minimized The inventor of the present invention recognized that an improved surgical tool includes the handpiece 14 integrated with the anoscope 51, such that the electrode 36, 38 distal ends are positioned in the slot 53 of the anoscope 51 to make contact with the hemorrhoid 50, without requiring the surgeon 52 to maintain a fixed relative position between the handpiece 14 and the anoscope 51.

As previously discussed, the probe base 48 in a conventional surgical tool 10 is constructed to be received in the aperture 40 at four orientations (90° rotations), selected by the surgeon 52 depending upon the rectal quadrant to be treated. The inventor of the present invention recognized that the aperture 40 could be redesigned to permit finer increments in the orientation of the probe base 48 when received in the aperture 40, such as at 45° rotational increments, for example.

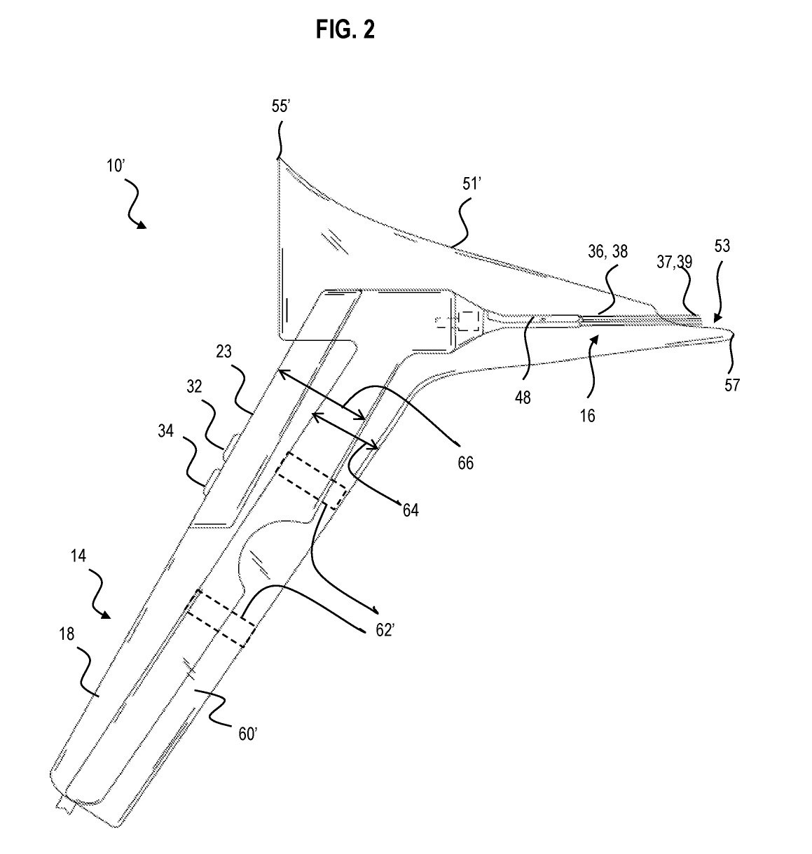

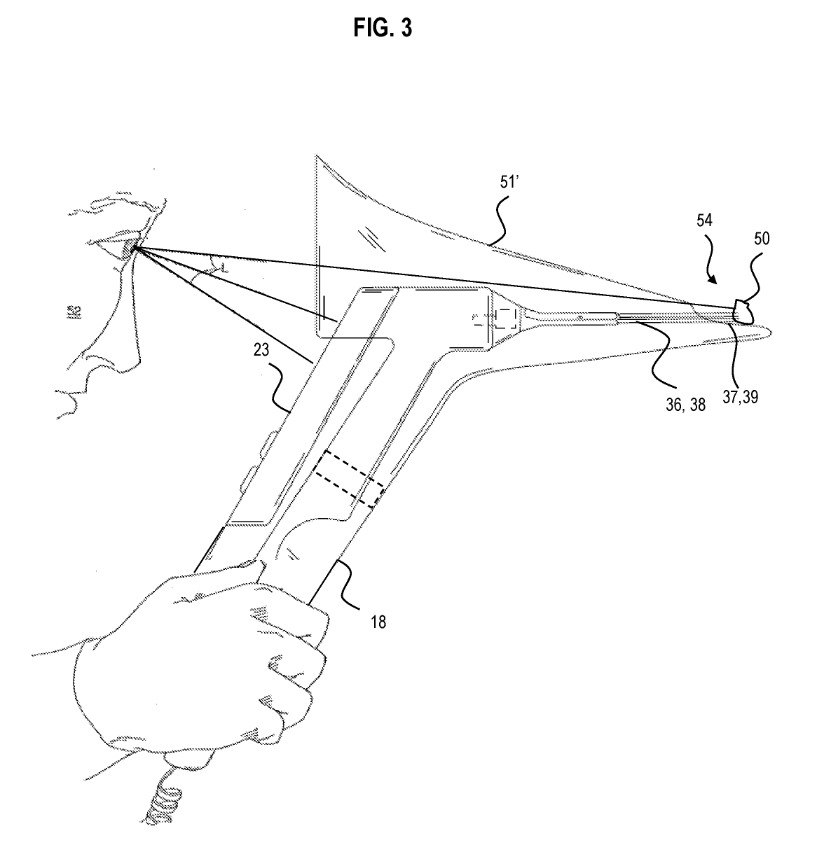

FIGS. 2-3 are side views of an improved surgical tool 10′ including an integrated probe 16 and anoscope 51′ for electrical current therapy to treat hemorrhoids 50, according to one embodiment of the present invention. Some numbered features of the surgical tool 10′ are similar to the equivalent numbered features of the surgical tool 10 discussed above and thus these numbered features require no further discussion herein. The description of this embodiment of the present invention focuses on structural distinctions between the surgical tool 10 and the surgical tool 10′.

As depicted in FIGS. 2-3, the proximal end 55′ of the anoscope 51′ includes a cavity 60′ that is sized to receive a portion of the handpiece 14. A depth 64 of the cavity 60′ is less than a depth 66 of the handpiece 14, so that the portion of the handpiece 14 is received within the cavity 60′. However, in other embodiments, the depth of the cavity may be equal to or greater than the depth 66 of the handpiece 14, such that the cavity 60′ is sized to receive all of the handpiece 14.

The handpiece 14 (and probe 16) are removably attached to the anoscope 51′. In the embodiment of FIGS. 2-3, a pair of fasteners 62′ are positioned along each side of the cavity 60′, to removably attach the handpiece 14 within the cavity 60′ of the anoscope 51′. Although FIGS. 2-3 depict a pair of fasteners on each side of the cavity 60′, less or more than two fasteners may be positioned on each side of the cavity 60′. In an example embodiment, the fasteners are one of magnetic fasteners, snap fasteners, or any type of fastener known to one skilled in the art. In one embodiment, the fasteners 62′ are configured such that the handpiece 14 and anoscope 51′ are fixedly attached together when the handpiece 14 is inserted into the cavity 60′. However, the handpiece 14 and anoscope 51′ can be detached from one another upon pulling the handpiece 14 from the cavity 60′ with a sufficient force that exceeds a predetermined threshold.

As shown in FIGS. 2-3, the cavity 60′ and fasteners 62′ are configured such that when the handpiece 14 is removably attached to the anoscope 51′, distal ends 37, 39 of the probe electrodes 36, 38 are positioned within the slot 53 at a distal end 57 of the anoscope 51′ for electricity-conducting engagement with hemorrhoidal tissue 50. As a result, the relative position between the handpiece 14 and the anoscope 51′ that is required for electricity-conducting engagement between the electrode distal ends 37, 39 and the hemorrhoidal tissue 50 is maintained by the removable attachment between the handpiece 14 and the anoscope 51′. Consequently, as shown in FIG. 3, the surgeon 52 can perform the electrical current therapy with one hand that is used to hold the integrated handpiece 14 and anoscope 51′, while the surgeons 52 remaining hand is free. In one embodiment, the surgeon 52 activates the display elements on the planar face surface 23 with the same hand that is used to hold the integrated handpiece 14 and anoscope 51′. In another embodiment, the surgeon 52 activates the display elements on the planar face surface 23 with the other hand that is not used to hold the integrated handpiece 14 and anoscope 51′.

Additionally, as shown in FIG. 3, the handpiece 14 is removably attached to the anoscope 51′, such that the line of sight L of the surgeon 52 to the treatment site 54 is maintained and is not blocked by the handpiece 14. Additionally, the line of sight L of the surgeon 52 to the display elements on the planar face surface 23 of the handpiece 14 is maintained.

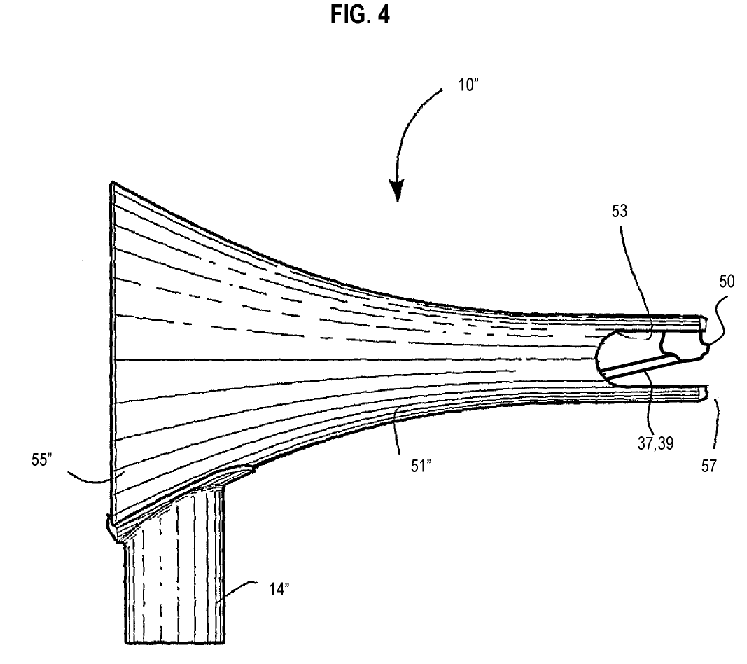

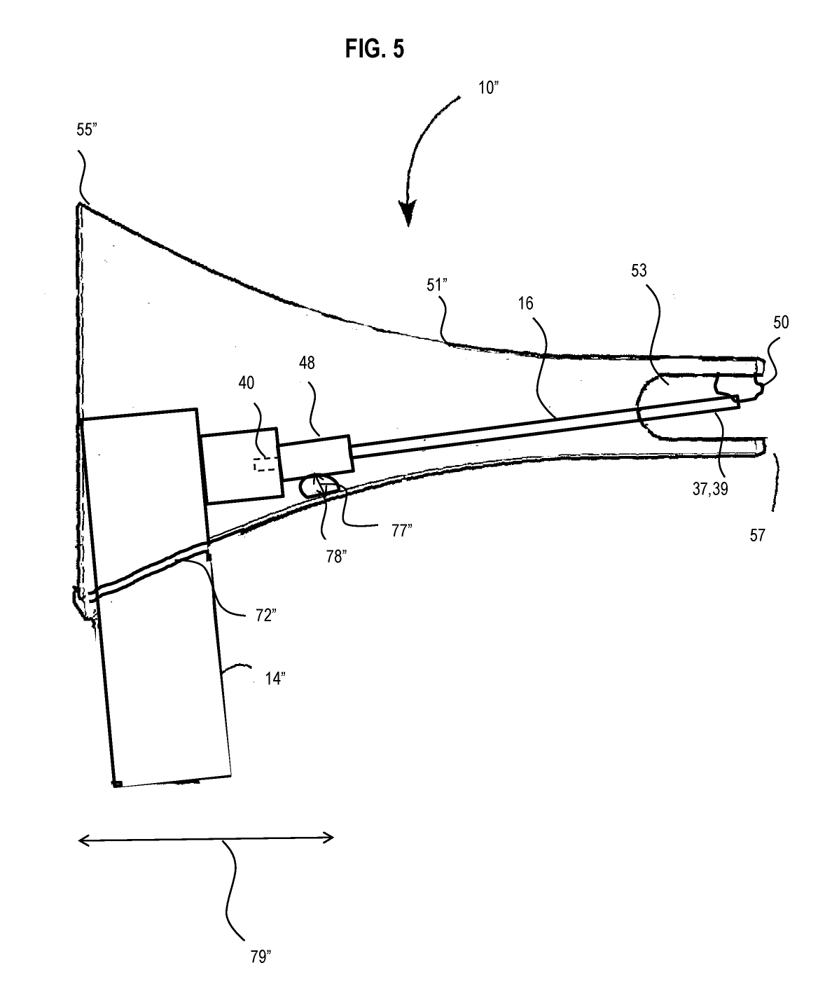

FIGS. 4-5 are side views of an improved surgical tool 10″ including an integrated probe 16 and anoscope 51″ for electrical current therapy to treat hemorrhoids 50, according to another embodiment of the present invention. Some numbered features of the surgical tool 10″ are similar to the equivalent numbered features of the surgical tool 10 discussed above and thus these numbered features require no further discussion herein. The description of this embodiment of the present invention focuses on structural distinctions between the surgical tool 10 and the surgical tool 10″.

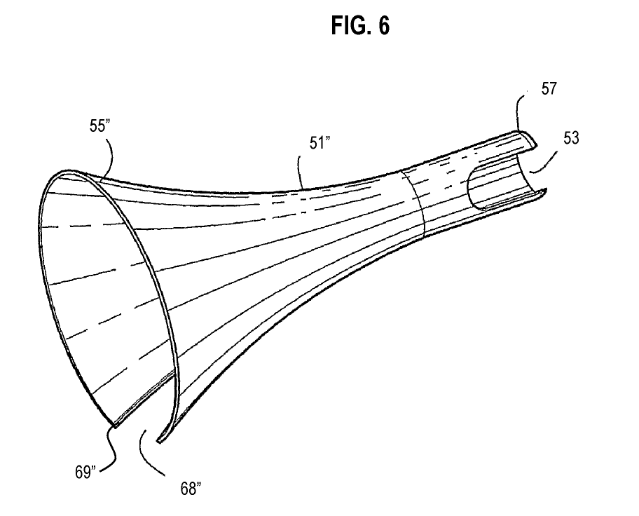

The anoscope 51″ has a somewhat conical shape where the proximal end 55″ is larger in diameter than the distal end 57. In an example embodiment, the anoscope 51″ is approximately 4.75 inches long or in a range of 4-5 inches, a diameter of the anoscope 51″ at the proximal end 55″ is approximately 2 inches or in a range of 1.5-2.5 inches and the diameter of the anoscope 51″ at the distal end 57 is approximately 0.89 inches or in a range of 0.5-1 inch. As shown in FIG. 6, the anoscope 51″ includes a slot 68″ near the proximal end 55″. In an example embodiment, the slot 68″ has a length of 1.25 inches or within a range of 1-3 inches and a width of 0.5 inches or within a range of 0.25-0.75 inches. However, these dimensions are merely exemplary and the dimensions of the anoscope 51″ are not limited to any specific numerical range.

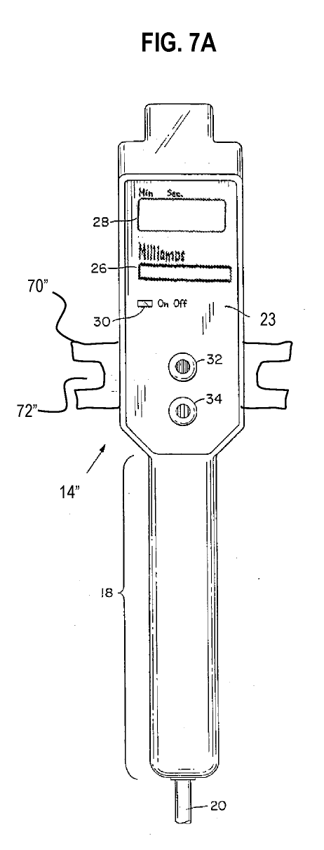

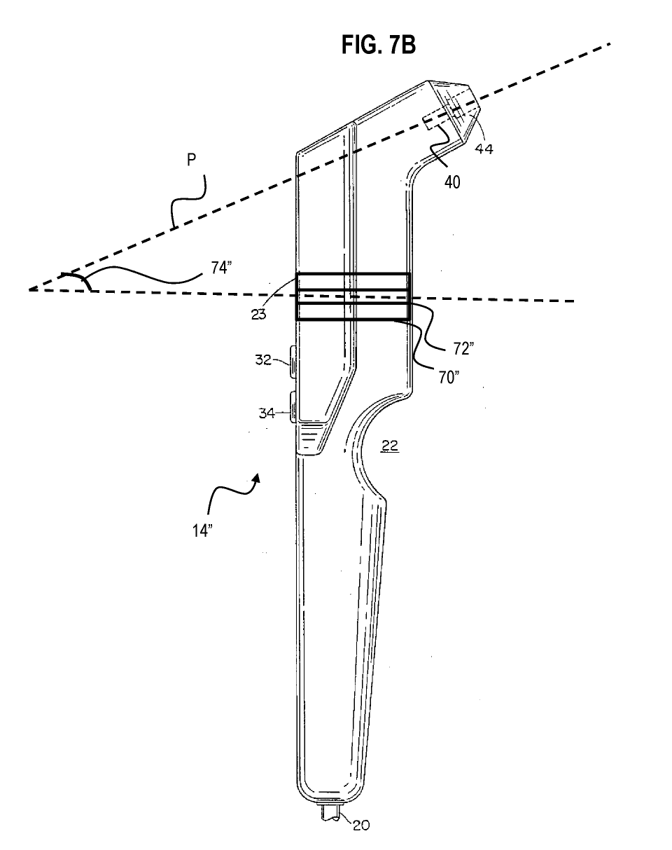

As shown in FIG. 7A, the handpiece 14″ includes a flange 70″ extending around a periphery of the handpiece 14″, where the flange 70″ includes a groove 72″ that is sized to engage a perimeter 69″ of the slot 68″. In an example embodiment, the flange has a length in a range of 0.25-1 inches and a width in a range of 0.25-1 inches (where the length and width are within the plane of FIG. 7A). In one embodiment, the flange 70″ and groove 72″ extend around an entire periphery of the handpiece 14″. In an example embodiment, the flange 70″ and groove 72″ extend around the periphery of the handpiece 14″ by a length equal to the perimeter 69″ of the slot 68″. In another embodiment, the flange and groove 72″ extends around a portion of the periphery of the handpiece 14″. The handpiece 14″ is removably attached to the anoscope 51″, by sliding the handpiece 14″ into the slot 68″ such that the groove 72″ engages the perimeter 69″ of the slot 68″. In one embodiment, the groove 72″ and the perimeter 69″ are shaped such that the perimeter 69″ and groove 72″ are fixedly attach together when the handpiece 14″ is slid into the slot 68″. However, the groove 72″ and the perimeter 69″ can be detached from one another upon pulling the handpiece 14″ from the slot 68″ with a sufficient force that exceeds a predetermined threshold.

As shown in FIGS. 4-5, the slot 68″ and groove 72″ are configured, such that upon removably attaching the handpiece 14″ to the anoscope 51″, the distal ends 37, 39 of the electrodes 36, 38 are positioned within the slot 53 at the distal end 57 of the anoscope 51″ for electricity-conducting engagement with hemorrhoidal tissue 50. As shown in FIG. 7B, the flange 70″ is disposed at an angle 74″ relative to the axis P of the probe 16 mounted in the aperture 40 at the distal end 44 of the handpiece 14″. In one embodiment, the angle 74″ is selected such that the distal ends 37, 39 of the probe electrode 36, 38 are oriented in the slot 53 at the distal end 57 of the anoscope 51″ upon engagement of the groove 72″ with the perimeter 69″ of the slot 68″. In an example embodiment, the angle 74″ is in a range of 0-90 degrees and more specifically is in a range of 30-60 degrees.

As further shown in FIG. 5, a mount 78″ is positioned along an inner surface of the anoscope 51″ between the proximal end 55″ and the distal end 57. In an example embodiment, the mount 78″ is separated from the proximal end 55″ by a distance 79″ in a range of 1-3 inches and has a height 77″ in a range of 0.1-1 inch and more preferably 0.25-0.5 inches. When the handle 14″ is removably attached in the slot 68″ and the groove 72″ engages the perimeter 69″ of the slot 68″, the mount 78″ provides support to the probe base 48, to align the distal ends 37, 39 of the probe electrode 36, 38 in the slot 53. Although FIG. 5 depicts the mount 78″ used to align the distal ends 37, 39 of the probe electrode 36, 38 in the slot 53, the embodiment of the present invention need not include the mount 78″, such that the engagement of the groove 72″ with the perimeter 69″ aligns the distal ends 37,39 of the probe electrode 36, 38 in the slot 53, without the need for the mount.

Although FIGS. 4-5 depict that the slot 68″ is oriented 90 degrees relative to the slot 53, the embodiment is not limited to this structural arrangement and the slot 68″ can be oriented to be aligned with the slot 53 or the slot 68″ can be oriented at an angular position relative to the slot 53 other than 90 degrees, such as 180 degrees, for example.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference to the extent they are not inconsistent with the teachings herein.

What is claimed is:

1. A surgical tool (10′, 10″) for use in surgical removal of hemorrhoidal tissue (50), said surgical tool comprising:
an anoscope (51′, 51″) with a proximal end (55′, 55″) and a distal end (57), and further having a slot (53) near said distal end (57) to receive the hemorrhoidal tissue (50); and
a probe (16) for delivering electrical current to the hemorrhoidal tissue (50), said probe including a base (48) with a distal end and a proximal end and at least one elongated probe electrode (36, 38) extending from said distal end of the base;
wherein the probe (16) is removably attached to the anoscope (51′, 51″) such that a distal end (37, 39) of the at least one elongated probe electrode (36, 38) is positioned within the slot (53) for electricity-conducting engagement with said hemorrhoidal tissue (50).

2. The surgical tool in accordance with claim 1 further comprising an instrument handpiece (14, 14″) wherein the proximal end of the base (48) is removably attached to the instrument handpiece (14) and wherein the instrument handpiece is removably attached to the anoscope (51′, 51″).

3. The surgical tool in accordance with claim 2 wherein the proximal end (55′) of the anoscope (51′) includes a cavity (60′) sized to receive a portion of the instrument handpiece (14) and wherein the portion of the instrument handpiece (14) is removably attached within the cavity.

4. The surgical tool in accordance with claim 3 further comprising at least one fastener (62′) within the cavity (60′) to removably attach the instrument handpiece (14) within the cavity (60′).

5. The surgical tool in accordance with claim 3 wherein a depth (64′) of the cavity (60′) is less than a depth (66′) of the handpiece (14).

6. The surgical tool in accordance with claim 2 wherein the anoscope (51″) has a somewhat conical shape wherein said proximal end (55″) is larger in diameter than said distal end (57), and wherein the anoscope (51″) includes a slot (68″) near said proximal end (55″) and wherein the handpiece (14″) includes a flange (70″) extending around a periphery of the handpiece (14″), said flange (70″) including a groove (72″) configured to engage the slot (68″) near said proximal end (55″) of the anoscope (51″) to removably attach the handpiece (14″) to the anoscope (51″).

7. The surgical tool in accordance with claim 6 wherein a distal end (44) of the handpiece (14″) includes an aperture (40) to receive the proximal end of the base (48) and wherein the flange (70″) is disposed at an angle (74″) relative to an axis (P) of the probe (16) mounted to the distal end (44) of the handpiece (14″).

8. The surgical tool in accordance with claim 7 wherein the angle (74″) is selected such that the distal end (37, 39) of the probe electrode (36, 38) is oriented in the slot (53) at the distal end (57″) of the anoscope (51″) upon engagement of the groove (72″) within the slot (68″) near the proximal end (55″) of the anoscope (51″).

9. The surgical tool in accordance with claim 6 further comprising a mount (78″) positioned along an inner surface of the anoscope (51″) between the proximal end and the distal end (55″, 57″), wherein the mount is configured to provide support to the probe base (48) and align the distal end (37, 39) of the probe electrode (36, 38) in the slot (53) at the distal end of the anoscope.

10. The surgical tool in accordance with claim 6 wherein the slot (53) near said distal end (57) of the anoscope (51″) is oriented 180 degrees from the slot (68″) near said proximal end (55″) of the anoscope.

11. A surgical tool (10′, 10″) for use in surgical removal of hemorrhoidal tissue (50), said surgical tool comprising:
an anoscope (51′, 51″) with a proximal end (55′, 55″) and a distal end (57), and further having a slot (53) near said distal end (57) to receive the hemorrhoidal tissue (50);
a probe (16) including at least one elongated probe electrode (36, 38) for delivering electrical current to the hemorrhoidal tissue (50); and
an instrument handpiece (14, 14″) wherein the probe (16) is removably attached to the instrument handpiece (14, 14″) and wherein the instrument handpiece is removably attached to the anoscope (51′, 51″) such that a distal end (37, 39) of the at least one elongated probe electrode (36, 38) is positioned within the slot (53) for electricity-conducting engagement with said hemorrhoidal tissue (50).

12. The surgical tool in accordance with claim 11, wherein the proximal end (55′) of the anoscope (51′) includes a cavity (60′) sized to receive a portion of the instrument handpiece (14) and wherein the portion of the instrument handpiece (14) is removably attached within the cavity.

13. The surgical tool in accordance with claim 12 further comprising at least one fastener (62′) within the cavity (60′) to removably attach the instrument handpiece (14) within the cavity (60′).

14. The surgical tool in accordance with claim 12 wherein a depth (64′) of the cavity (60′) is less than a depth (66′) of the handpiece (14).

15. The surgical tool in accordance with claim 11 wherein the anoscope (51″) includes a slot (68″) near said proximal end (55″) and wherein the handpiece (14″) includes a flange (70″) extending around a periphery of the handpiece (14″), said flange (70″) including a groove (72″) configured to engage the slot (68″) to removably attach the handpiece (14″) to the anoscope (51″).

16. The surgical tool in accordance with claim 15 wherein the flange (70″) is disposed at an angle (74″) relative to an axis (P) of the probe (16) mounted to the handpiece (14″).

17. The surgical tool in accordance with claim 16 wherein the angle (74″) is selected such that the distal end (37, 39) of the probe electrode (36, 38) is oriented in the slot (53) at the distal end (57″) of the anoscope (51″) upon engagement of the groove (72″) within the slot (68″).

18. The surgical tool in accordance with claim 15 further comprising a mount (78″) positioned along an inner surface of the anoscope (51″) between the proximal end and the distal end (55″, 57″), wherein the mount is configured to provide support to the probe (16) and align the distal end (37, 39) of the probe electrode (36, 38) in the slot (53) at the distal end of the anoscope.

19. The surgical tool in accordance with claim 15 wherein the slot (53) near said distal end (57) of the anoscope (51″) is oriented 180 degrees from the slot (68″) near said proximal end (55″) of the anoscope.

20. The surgical tool in accordance with claim 13, wherein the fastener (62′) is one of a magnetic fastener and a snap fastener.
